(19) United States
(12) Patent Application Publication
(10) Pub. No.: US 2021/0106740 A1
(43) Pub. Date: Apr. 15, 2021

(54) Holder For Medical Treatment Apparatuses, Module and Treatment Apparatus

(71) Applicant: Fresenius Medical Care Deutschland GmbH, Bad Homburg (DE)

(72) Inventors: Juergen Klewinghaus, Oberursel (DE); Alexander Schroers, Frankfurt am Main (DE)

(21) Appl. No.: 16/498,619

(22) PCT Filed: Mar. 28, 2018
(86) PCT No.: PCT/EP2018/057935

§ 371 Date: Sep. 27, 2019

(30) Foreign Application Priority Data
Mar. 30, 2017 (DE) 10 2017 106 910.0

(51) Int. Cl. A61M 1/16; A61M 1/34

(52) U.S. Cl. CPC A61M 1/1652; A61M 2202/0208; A61M 2209/082; A61M 1/34

(57) ABSTRACT

The present disclosure relates to a holder for releasably coupling at least one external medical module, for example a blood filter, to a medical treatment apparatus, wherein the holder comprises at least a first reception unit and a second reception unit.

### CROSS-REFERENCE TO RELATED APPLICATIONS

The present application is the national stage entry of International Patent Application No. PCT/EP2018/057935, filed on Mar. 28, 2018, and claims priority to Application No. DE 10 2017 106 910.0, filed in the Federal Republic of Germany on Mar. 30, 2017, the disclosure of which are expressly incorporated herein in entirety by reference thereto.

### TECHNICAL FIELD

The present disclosure relates to a holder for a medical treatment apparatus. It further relates to a treatment apparatus, and a medical module or a medical functional device.

### BACKGROUND

Holders can be used to releasably mount modules like, filters, gas exchangers, absorbers, or the like to an apparatus for treating a patient.

### SUMMARY

This document describes holders for medical treatment apparatuses. In addition, a medical module, which can be a disposable, and a treatment apparatus are described herein.

A holder for releasably coupling or mounting at least one external medical module, such as a blood filter, to a medical treatment apparatus, in particular to a blood treatment apparatus, is described herein as having at least a first reception unit and a second reception unit.

The medical module disclosed herein, in particular the medical module embodied as a dialysis filter or a dialyzer, is connected to at least a first holder and to at least a second holder disclosed herein.

The treatment apparatus disclosed herein comprises two holders.

In the embodiments described herein, the use of the expression “may be” and “may have” etc. is synonymous with “is preferably” or “has preferably,” etc. respectively, and is intended to illustrate an embodiment according to the present disclosure.

Embodiments disclosed herein may comprise one or several of the aforementioned or following features. The features mentioned herein may, in any combination thereof, be subject-matter of the embodiments disclosed herein unless the person skilled in the art recognizes a specific combination as technically impossible. Embodiments disclosed herein are also subject-matter of the dependent claims.

Whenever numerical words are mentioned herein, the person skilled in the art will comprehend them as indications of numerical lower limits. Unless it leads the person skilled in the art to an evident contradiction, the person skilled in the art will, for example, comprehend the specification of “one” as encompassing “at least one”. This understanding is also equally encompassed by the present disclosure as the interpretation that a numeric word, for example, “one” may alternatively mean “exactly one”, wherever this is evidently technically possible to a person skilled in the art. Both interpretations are encompassed by the present disclosure and apply to all numerical words used herein.

Whenever an embodiment is disclosed herein, it represents an exemplary embodiment.

The information “top” and “bottom” are to be understood, in case of doubt as seen by the person skilled in the art, as absolute or relative spatial information which refers to the position and/or orientation of the respective component during its intended use.

When “module” is mentioned herein, it can be understood e.g. as the first module. In the present disclosure, a module can be a medical functional device, a filter, a blood filter, a cartridge, an adapter, a gas exchanger, an adsorber, an absorber, or another device utilized in the treatment of a patient. A module may be a disposable or a non-disposable.

In some embodiments, the holder comprises no locking device to lock against a rotation of the first reception unit against the second reception unit.

In some embodiments described herein, the module is not a device, and is particularly not a device having electronic components.

In some embodiments described herein, the module is not a tube, particularly not a flexible tube, and/or comprises no such tube.

Optionally, the module may be connected to a tube or integrally manufactured with a tube. In some embodiments described herein, the module is not retained in the holder by or with its—integral or connected—tube section.

In some embodiments described herein, the module is not tube-shaped and/or is not flexible.

In some embodiments described herein, the module comprises a cylindrical shape. The module may comprise an exterior pipe section, which is closed at one or both front/end faces by a cover. The front/end faces and/or the cover may comprise through-openings at which tubes may be arranged.

In some embodiments described herein, the module is not longer than 40 cm.

In some embodiments described herein, the holder is not connected to a device designed to be fixed at a blood treatment apparatus. The device may be a cover or a covering device—e.g. for a surface of a treatment apparatus—which is provided for fixing cartridges, filters or the like thereon. The device may be made of plastic. The device may be large. The device may comprise sections made of plastic film. The device may comprise through-openings to be fixed on or in front of a surface of the treatment apparatus.

In some embodiments described herein, the holder is not a multi-part holder.

In some embodiments described herein, the holder comprises no U-shaped elements, and, in particular, comprises no U-shaped frame elements

In some embodiments described herein, the first reception unit comprises a clamp, or a clamping holder, having clamp jaws and optionally a closing element. In some embodiments described herein, the clamp jaws are movable relative to each other and/or are arranged to be movable from a first position relative to each other to a second position relative to each other.

In some embodiments described herein, the second reception unit is, or comprises, a fixing section. In some examples, the fixing section serves the fixing, in particular releasable fixing, of the holder on or to a section of the medical treatment apparatus, in particular on a bar section of the medical treatment apparatus.

In some embodiments described herein, the holder comprises a third reception unit in addition to the first reception unit and the second reception unit.

In some embodiments described herein, both the first reception unit and the second reception unit of the holder comprise a clamp having clamp jaws and an optional closing element. In some examples, the clamp jaws are movable relative to each other and/or are arranged to be movable from a first position relative to each other to a second position relative to each other.

In some embodiments described herein, the first reception unit and the third reception unit—and optionally also the second reception unit—of the holder each comprise at least one clamp with clamp jaws (or with clamp sections not embodied as jaws) and, optionally, an optional closing element. In some examples, the clamp jaws are, exemplarily and without being limited thereto, movable relative to each other while being assisted by the optional closing element, and/or arranged to be movable from a first position relative to each other to a second position relative to each other.

In some embodiments described herein, a rotating section is arranged between the first reception unit and the second reception unit of the holder and/or between the first reception unit and the third reception unit of the holder. Said rotating section allows for rotation of the first reception unit relative to the second reception unit or rotation of the first reception unit relative to the third reception unit. The rotating section may configured to allow for rotation about a rotation axis, or about two or more rotation axes.

In some embodiments described herein, the first reception unit and the second reception unit of the holder are identical. In some embodiments described herein, the first reception unit and the third reception unit of the holder are identical. In some embodiments described herein, the second and the third reception unit of the holder are identical. In some embodiments described herein, the first, the second and the third reception unit of the holder are identical.

In some embodiments described herein, a straight line runs through the first reception unit and the third reception unit. A straight line preferably does not run through the second reception unit as well. Alternatively, the straight line runs through the second reception unit as well as the first and third reception units. In some embodiments described herein, there is a separation between the first reception unit and the third reception unit, preferably along the straight line. In some examples, the separation between the first reception unit and the third reception unit is at least as long as the width of the first reception unit, preferably in a direction along the straight line.

In some embodiments described herein, the holder comprises a support structure for supporting the holder. In some embodiments described herein, the first reception unit of the holder is arranged between the support structure and the second reception unit of the holder. In some embodiments described herein, the support structure is arranged at the first reception unit of the holder and/or at the second reception unit of the holder. The support structure may be provided at the second holder, but also at any other holder.

In some embodiments described herein, at least both the first holder and the second holder are connected to the module. In some embodiments described herein, the second holder is connected to both the module and at least one further module or to an adapter for receiving a further module. In some embodiments described herein, the second holder is connected to the module both above and below the first reception unit of the first holder. In some embodiments described herein, the second holder is arranged to support itself with its support structure positioned against the first holder.

In some embodiments described herein, the medical treatment apparatus is connected to a first module via the first holder. In some examples, the first module is connected to at least a second holder, and the second holder is connected to a second module or to an adapter for receiving a second module. In some examples, the second module or the adapter is connected to a third holder.

In some embodiments described herein, the medical treatment apparatus is an apparatus for therapeutic apheresis, an apparatus for liver support therapy, an apparatus for oxygenation, or a blood treatment apparatus, in particular a dialysis apparatus, such as a hemofiltration apparatus or hemodialysis apparatus.

In some embodiments described herein, at least one or all holders are arranged between a section of the treatment apparatus and a module, or between a first module and a second module (or adapter for a respective second module). As such, in some embodiments described herein, the holder is not connected to further structure other than the ones mentioned supra, and/or is not supported by same.

In some embodiments described herein, the three modules are connected to the treatment apparatus by the holder(s), preferably in series or as a cascade.

In some embodiments described herein, there are more than three holders provided.

In some embodiments described herein, a reception unit is a fixing section.

In some embodiments described herein disclosed herein “receiving” refers to holding or fixing or connecting.

In some embodiments described herein, the holder comprises at least a connecting section by means of which one or more reception units are held at or connected to each other at the remaining holders.

In some embodiments described herein, the connecting section or another section of the holder comprises a device by means of which one or more receptions are releasably connected to further sections of the holder, and are preferably releasable by hand and/or without tools. The device by means of which one or more receptions are releasably connected may be a latching closure, a snap-in closure, a clamping closure, a click closure, a connector, or the like.

In some embodiments described herein, at least one reception of a holder is embodied and/or is used to be fixed, in particular releasably, to at least one reception unit of a second holder. The two reception units, from which one is taken up by the other, may be designed to match each other.

Some or all of the embodiments disclosed herein may encompass one, several or all of the aforementioned and/or following advantages.

One advantage of the present disclosure may be that a greater freedom is achieved for modules required for a patient's treatment to be releasably connected to a treatment apparatus used for the treatment. Modules for which no reception unit has been provided on the treatment apparatus or no reception unit could be provided due to lack of space, may still be indirectly fixed to the treatment apparatus as disclosed herein. As such, several modules may advantageously be indirectly fixed to the treatment apparatus in a cascade arrangement.

The required or desired stability of the releasable fixing of modules to the treatment apparatus may advantageously be ensured by the support structures disclosed herein.

If several of the utilized holders are identical to each other, then advantages may be achieved in the procurement of the holders amongst others.

An advantage of the fact that the holder may be disassembled, e.g. by the latching closure, snap-in closure, etc., may be that. an arbitrary first reception unit could be connected to an arbitrary second reception unit to form a holder by hand and/or using a simple mechanism.

The holder may thus be simply adapted to the concrete, momentary and possibly changing needs or requirements of the user.

### BRIEF DESCRIPTION OF THE FIGURES

The present disclosure is exemplarily described with regard to the enclosed drawings, in which identical reference numerals refer to the same or similar components. In the figures, the following is shown:

FIG. 1 shows a first embodiment of two holders which are directly or indirectly connected to a treatment apparatus.

FIG. 2 shows a holder from above at an angle (i.e., showing the top side).

FIG. 3 shows the arrangement from FIG. 1 with a variation.

FIG. 4 shows one of the embodiments illustrated in FIG. 3.

FIG. 4a shows an exemplary embodiment of a holder with two clamps.

FIG. 5 shows a rotating section arranged between a first reception unit and a second reception unit of the second holder of FIG. 4.

FIG. 6 shows a holder having a third reception unit.

FIG. 7 shows an embodiment of a holder having a support structure.

FIG. 8 shows an exemplary embodiment of FIG. 7 in which the support structure supports itself laterally against the holder.

FIG. 9 shows an exemplary embodiment of FIG. 7 and/or of FIG. 8 in which the support structure supports itself from the top against the first holder.

FIG. 10 shows a further embodiment of the present invention.

FIG. 11 shows a cascade arrangement of e.g. three holders which are arranged in a row.

FIG. 11a shows an exemplary embodiment of the second or third holder of FIG. 11.

FIG. 12 shows an exemplary embodiment of the holder.

### DETAILED DESCRIPTION OF THE FIGURES

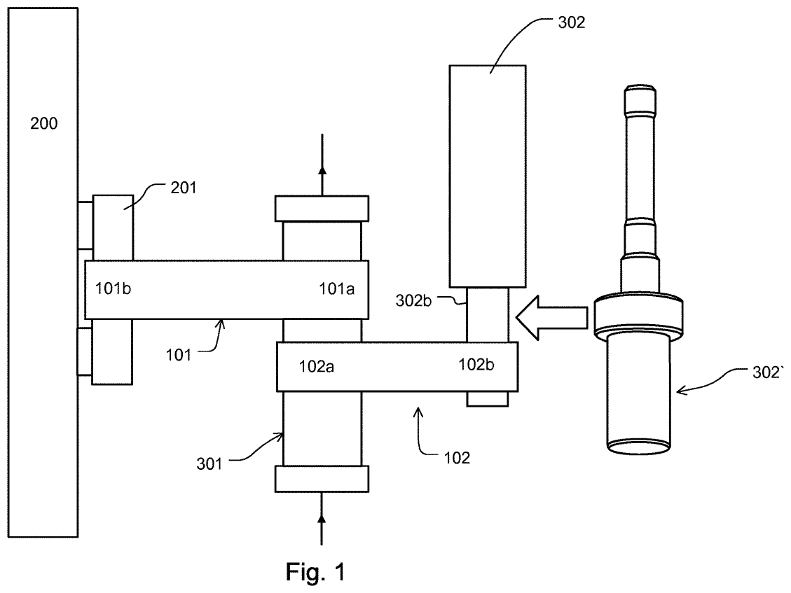

FIG. 1 shows a first embodiment of an example holder connected to a treatment apparatus 200.

The holder is shown in FIG. 1 once as a first holder 101 and once as a second holder 102. The first holder 101 comprises a first reception unit 101a and a second reception unit 101b. The second holder 102 comprises a first reception unit 102a and a second reception unit 102b.

The treatment apparatus 200 comprises a bar section 201 with which the first holder 101 is releasably connected via its second reception unit 101b. The first holder 101 is releasably connected via its first reception unit 101a to a first module 301, which may be, for example a dialyzer or blood filter.

The first module 301 is in turn releasably connected to the second holder 102 through the first reception unit 102a of the second holder 102. The first module 301 is thus used as an end point for the first holder 101 and is fixed by the first holder 101 to the treatment apparatus 200. At the same time, the first module 301 serves as support or starting point for the second holder 102, which in this way is not directly, but rather indirectly releasably connected to the treatment apparatus 200 via the first module 301.

The second holder 102 comprises a second reception unit 102b in addition to its first reception unit 102a with which it is fixed to the first module 301. The second reception unit 102b of the second holder 102 in the example of FIG. 1 serves as the reception of a gas exchanger as an example of a second module 302. The second module 302 could, however, be any other object and is not restricted to a gas exchanger. The second module 302 may, as in FIG. 1, optionally have the form of a cartridge.

The reference numeral 302 may instead of denoting the second module alternatively denote, for example, an adapter onto which a second module 302 may be mounted. For better clarity, an adapter 302′ is shown in addition to the schematically shown second module 302. The adapter may be used as a second module 302 and may itself serve the to receive further components, for example, as a gas exchanger, which may be inserted into said adapter 302.

The first reception units 101a and 102a and/or the second reception units 101b and 102b may optionally be identical. In this, the first holder 101 and the second holder 102 may be identical as a whole.

The section 302b of the second module 302, which is inserted into the second reception unit 102b, may advantageously have the same or similar diameter as the bar section 201. In such an instance, the first holder 101 and the second holder 102 may identical to each other anyway.

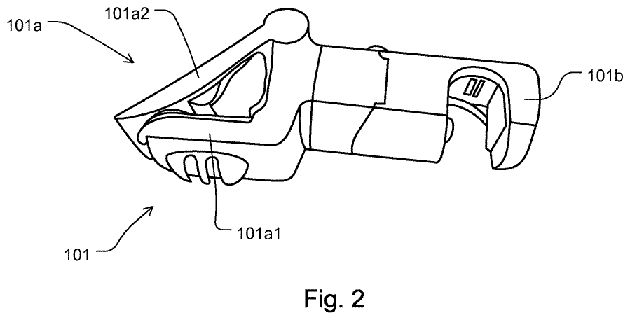

FIG. 2 shows a first holder 101.

The first reception unit 101a is a clamp with clamp jaws 101a1 and 101a2. A closing element in the form of a spring, a screw connection, a latching mechanism, or the like is provided, but not shown in FIG. 2.

The second reception unit 101b is optionally designed to receive the bar section 201 (as shown in FIG. 1). In some implementations, the second reception unit 101b is provided as a clamp. Likewise, the first reception unit 101a could be like the second reception unit 101b, and is not necessarily designed as a clamp.

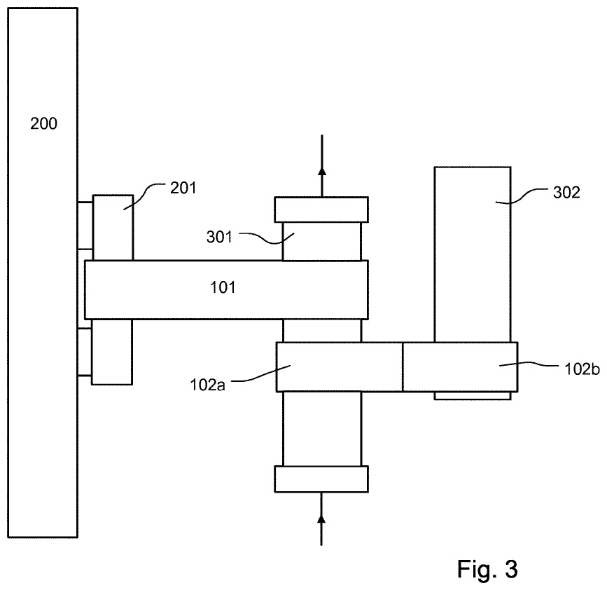

FIG. 3 shows the arrangement according to the embodiment depicted of FIG. 1 with a variation. That is, the second reception unit 102b of the holder 102 is an adapter which serves as the reception of a further component, such as a second module 302, a filter, a gas exchanger, an absorber, and the like. For this purpose, the second reception unit 102b of the second holder 102 may be designed like the first reception unit 102a, with which the second holder 102 is connected to the module 301.

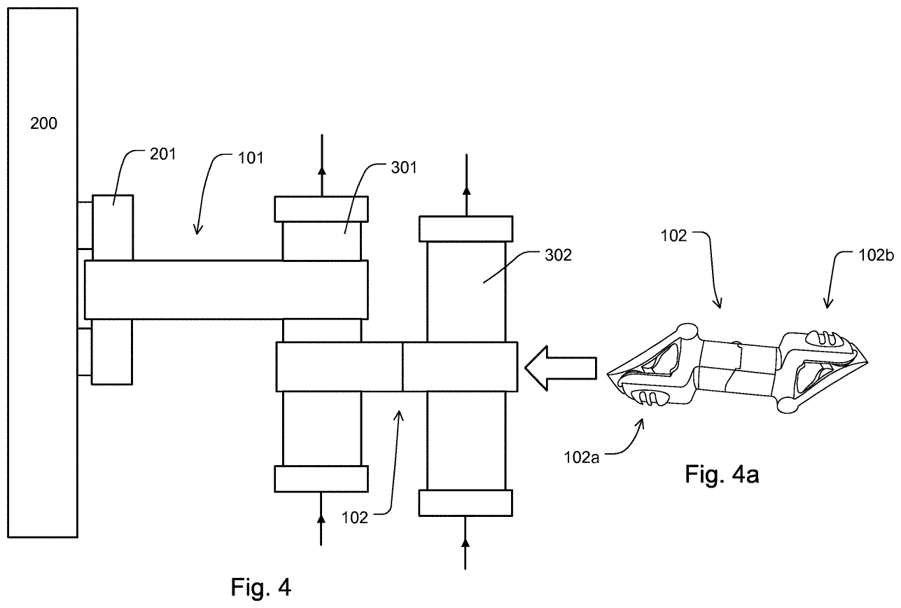

FIG. 4 shows an embodiment mentioned in FIG. 3. FIG. 4a shows a possible embodiment of the second holder 102. The second holder 102 shown in FIG. 4a comprises two clamps, as a first reception unit 102a and second reception unit 102b, which may optionally be identical.

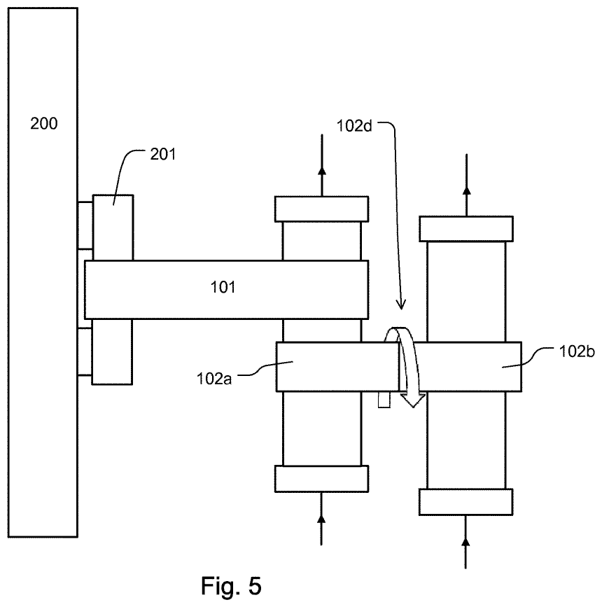

FIG. 5 shows, based on the embodiment illustrated in FIG. 4—without being restricted thereto—that a rotating section 102d can be provided between the first reception unit 102a and the second reception unit 102b of the second holder 102. The first reception unit 102a and the second reception unit 102b of the second holder 102 may be rotated relative to each other about rotating section 102d. Rotating section 102D may advantageously increase or extend the field of application of the second holder 102. The axis of rotation enabled by the rotating section 102d may, in an exemplary embodiment, extend through or parallel to the longitudinal direction of the second holder 102.

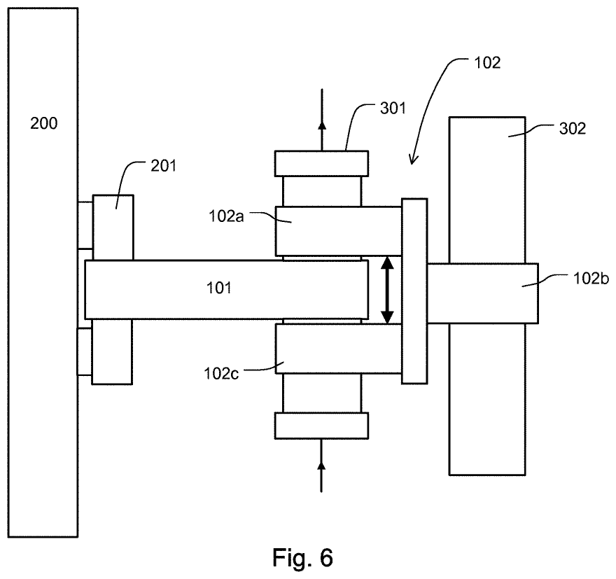

FIG. 6 shows, based on the illustration of FIG. 5 without being restricted thereto, that the second holder 102 may comprise a third reception unit 102c in addition to the first reception unit 102a and the second reception unit 102b. The first reception unit 102a and the third reception unit 102c shown in FIG. 6 are exemplarily identical. Both the first reception unit 102a and the third reception unit 102c are releasably connected to or receive the first module 301. At least in part due to the distance between them, together the first reception unit 102a and the third reception unit 102c increase the stability of the connection of the second holder 102 with the second module 302, connected thereto by the second reception unit 102b. The distance between the first reception unit 102a and the third reception unit 102c, which is designated by double arrows in FIG. 6, is optionally at least as large as the width of first reception unit 101a of the first holder 101 (as shown in FIG. 1).

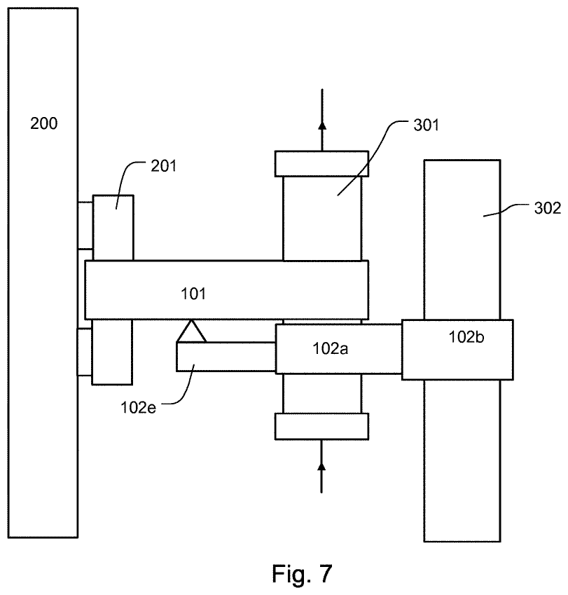

An increased stability of the connection between the second holder 102 and the second module 302 may also be achieved by the second holder 102 shown in FIG. 7. The second holder 102 in FIG. 7 comprises a support structure 102e in addition to the third reception unit 102c shown in FIG. 6. The support structure 102e is arranged in the example of FIG. 7 below the first holder 101 and may support itself against the first holder 101 from below the first holder 101. Whenever a support structure is mentioned herein, this may refer to a section specifically intended or configured for supporting. A support structure may optionally be a protrusion. A support structure may optionally be arranged so that it ends in an area of a plane extending through an upper surface of the second holder 102 or through of one of the receptions of the second holder 102, as shown in FIG. 7.

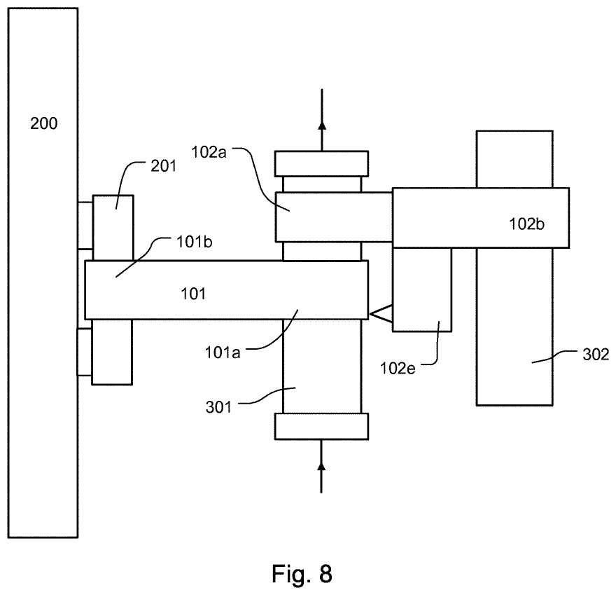

FIG. 8 shows an additional or alternative embodiment to that of FIG. 7, in which the support structure 102e laterally supports itself against a portion of the first holder 101, for example the first reception unit 101a. The support structure 102e could alternatively support itself against the first module 301 or against another structure. The support structure 102e may be position at an angle relative to the second holder 102, to the first reception unit 102a or to the second reception unit 102b of the second holder 102 For example, in the exemplary embodiment of FIG. 8, the support structure 102e is at a right angle relative to the longitudinal direction of the second holder 102.

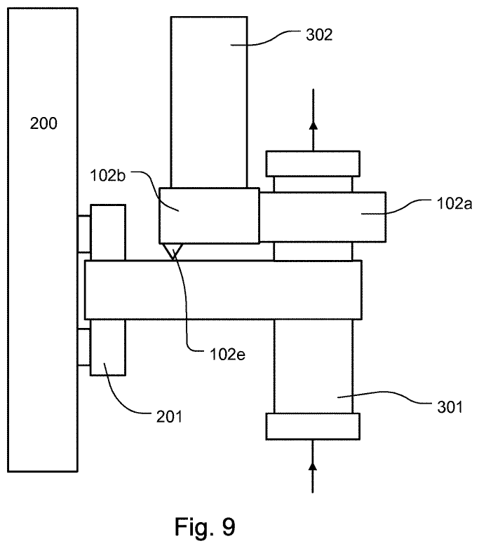

FIG. 9 shows an additional or alternative embodiment to the embodiments shown in FIG. 7 and/or FIG. 8. In FIG. 9, the support structure 102e supports itself from the top against the first holder 101, e.g. against the first reception unit 101a, the second reception unit 101b or a connecting section between the first reception unit 101a and the second reception unit 101b.

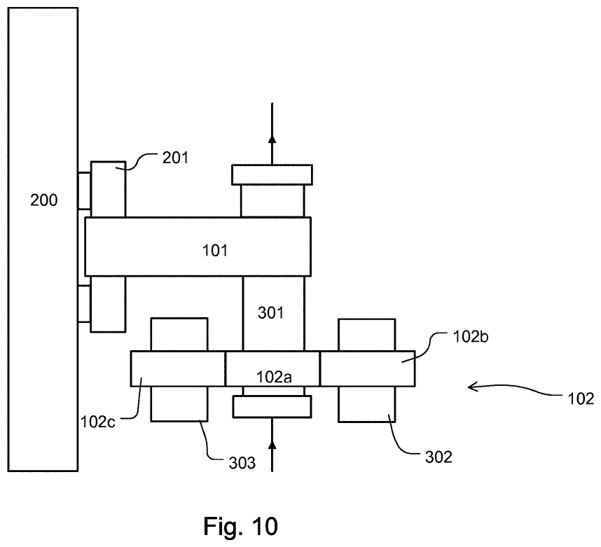

FIG. 10 shows a further embodiment of the present invention. In the embodiment depicted in FIG. 10, the first module 301 is connected to a first holder 101 and to a second holder 102. The second holder 102 of FIG. 10 comprises a first reception unit 102a, which is releasably connected to the first module 301, a second reception unit 102b, and a third reception unit 102c. Unlike in the embodiment of FIG. 6 in which the first reception unit 102a and the third reception unit 102c each receive the same first module 301 and therefore may be of identical design, the third reception unit 102c of FIG. 10 may not be of identical design as the first reception unit 102a, and may optionally be partially or exactly like the second reception unit 102b of FIG. 6 or FIG. 7. For example, the third reception unit 102c of FIG. 10 is provided and configured to receive a further module 303.

Alternatively, the first reception unit 102a may be embodied like the third reception unit 102c.

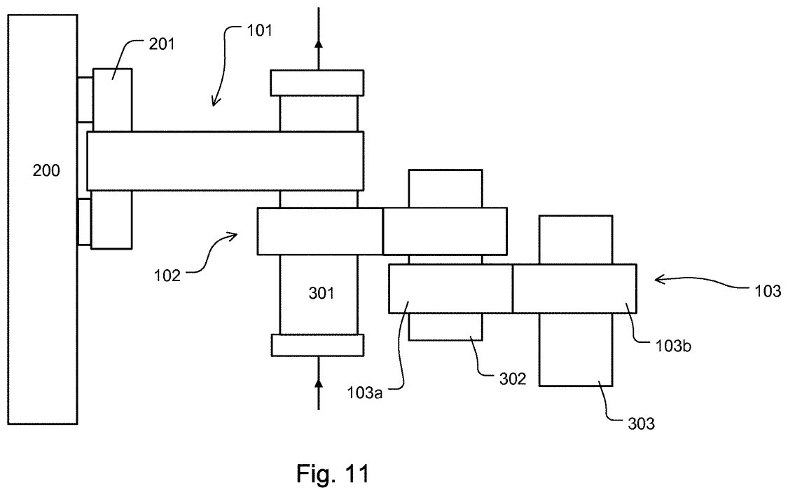

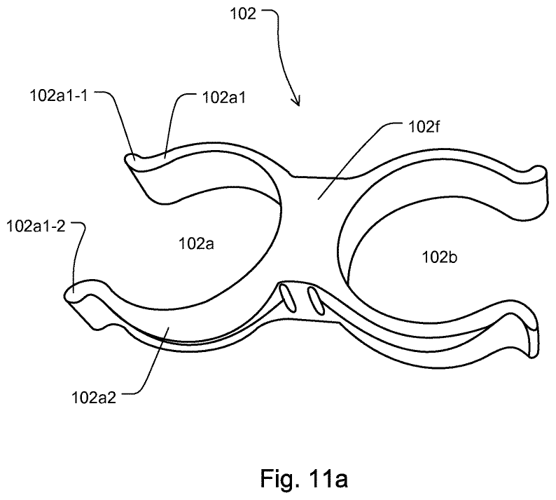

FIG. 11 shows a cascade arrangement of three example holders 101, 102 and 103, which are arranged in a row. Herein, in a row means that the first holder 101 holds the first module 301, the first module 301, e.g. a blood filter, holds the second holder 102, the second holder 102 holds a second module 302, e.g. a gas adsorber, the second module 302 also attaches to the third holder 103, which comprises a first reception unit 103a and a second reception unit 103b, and the third holder 103 finally holds a further module 303.

FIG. 11a shows an exemplary embodiment of the second holder 102 of FIG. 11. The second holder 102 may be similar or identical to the third holder 103 of FIG. 11. The second holder 102 of FIG. 11a comprises a first reception unit 102a, and a second reception unit 102b similar or identical to the first reception unit 102a. The first reception unit 102a and the second reception unit 102b are connected to each other by the connecting section 102f disposed between the first reception unit 102a and the second reception unit 102b. Both the first reception unit 102a and the second reception unit 102b are embodied in FIG. 11a as clamp sections. To this end, the first reception unit 102a of FIG. 11a includes a first clamp jaw 102a1 and a second clamp jaw 102a2. A closing element is not required in the embodiment in FIG. 11a since the clamping jaws 102a1, 102a2 are both elastic. The first clamp jaw 102a1 and second clamp jaw 102a2 in FIG. 11a move away from each other whenever the first reception unit 102a is, for example, moved onto a blood filter, and move towards each other, at least with their respective free ends 102a1-1 and 102a1-2, when the blood filter has been advanced past the free ends with its widest diameter. The first reception unit 102a of FIG. 11a includes two elastic sections together with the first clamp jaw 102a1 and the second clamp jaw 102a2. However, it may suffice in some cases to make only one of the two clamping jaws mentioned above elastic.

As shown in FIG. 11a, one or both of the free ends 102a1-1, 102a1-2 of the first and second clamp jaw 102a1, 102a2, respectively, may optionally extend away from the remaining part of the respective first or second clamp jaw 102a1, 102a2. The free ends 102a1-1, 102a1-2 may optionally be designed to define a curvature opposite the curvature of the main section of the respective clamp jaw 102a1, 102a2. The main section of the clamp jaws 102a1, 102a2 may facilitate the insertion of, for example, a blood filter into the first reception unit 102a. In addition, free ends 102a1-1, 102a1-2 may serve as a handle for actuation by the user.

As stated above, the second reception unit 102b may be designed like the first reception unit 102a.

The second holder 102 may consist of the first reception unit 102a, the second reception unit 102b and the connecting section 102f The connecting section 102f of FIG. 11a may be—like connecting sections of other embodiments—two-piece or multiple-piece, and may comprise a corresponding device by means of which the pieces of the connecting section 102f are releasably connected. The pieces of the connecting section 102f are preferably releasable by hand and/or without tools. Such a device by means of which the pieces of the connecting section 102f are releasably connected may be a latching closure, a snap-in closure, a clamping closure, a click closure, or the like.

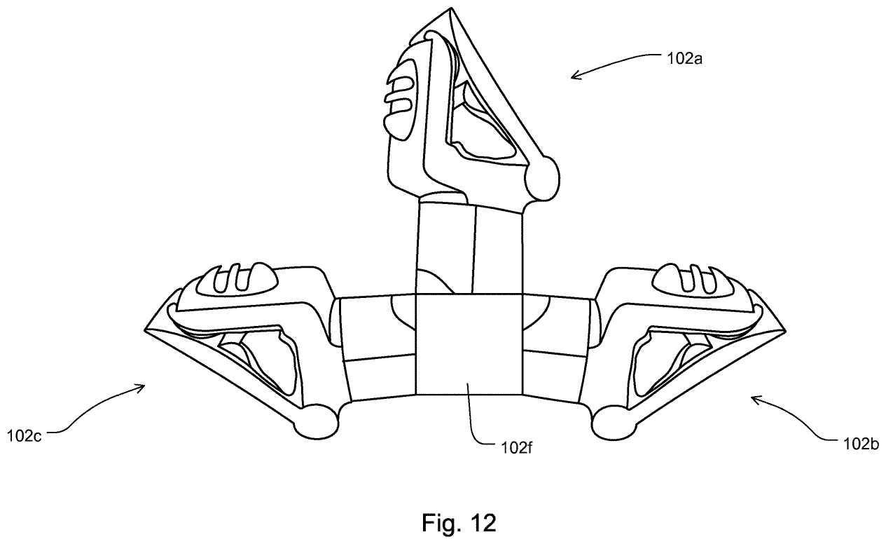

FIG. 12 shows a further embodiment of the holder disclosed herein. For example, the second holder 102 of FIG. 12 comprises three clamps, as a first reception unit 102a, second reception unit 102b and third reception unit 102c. The three clamps may optionally be identical.

As show in FIG. 12, the three reception units 102a, 102b, 102c may be connected to each other by a connecting section 102f The connecting section 102f may itself be equipped with a function. For example, the connecting section 102f may be a reception unit configured to receive an adapter.

Instead of comprising three clamps, the holder of FIG. 12 may also comprise more clamps. Instead of comprising only clamps, one or more of the reception units of FIG. 12 may designed differently. For example, one or more of the reception units of FIG. 12 may be designed like the second reception unit 101b of FIG. 2. The holder of FIG. 12 may be used in the arrangement of FIG. 10.

The connecting section 102f depicted in FIG. 12 may, like that of FIG. 11a, comprise a device which releasably holds together connecting sections or individual receptions (such as reception units 101a, 102a), etc.

### LIST OF REFERENCE NUMERALS

101 first holder
101a first reception unit
101a1 clamp jaw, first clamp jaw
101a2 clamp jaw, second clamp jaw
101b second reception unit
102 second holder
102a first reception unit
102a1 clamp jaw, first clamp jaw
102a2 clamp jaw, second clamp jaw
102a1-1 free end
102a1-2 free end
102b second reception unit
102c third reception unit
102d rotating section
102e support structure
102f connecting section
103 third holder
103a first reception unit
103b second reception unit
200 treatment apparatus
201 bar or bar section
301 first module
302 second module, or adapter for a second module
302b section of the second module or adapter
302′ adapter
303 further module, or adapter for a further module

1.-19. (canceled)

20. A holder for releasably coupling at least one external medical module to a medical treatment apparatus wherein the holder comprises a first reception unit and a second reception unit.

21. The holder of claim 20, wherein
the first reception unit comprises a clamp, wherein the clamp comprises a pair of clamp jaws, and wherein each of the clamp jaws are movable relative to each other.

22. The holder of claim 21, wherein the clamp further comprises a closing element.

23. The holder of claim 20, wherein the second reception unit comprises a fixing section, wherein the fixing section releasably fixes the holder on a section of the medical treatment apparatus.

24. The holder of claim 20, wherein the holder further comprises a third reception unit.

25. The holder of claim 24, wherein:
two or more reception units selected from the group consisting of the first reception unit, the second reception unit and the third reception unit each comprise a clamp with clamp jaws, and
at least one of the clamp jaws of each respective clamp is movable relative to the other one of the clamp jaws.

26. The holder of claim 25, wherein the two or more reception units further comprise a closing element.

27. The holder of claim 24, further comprising a rotating section arranged between two reception units of the holder selected from the group consisting of the first reception unit, the second reception unit, and the third reception unit, wherein the rotating section enables a rotation of a first selected reception unit relative to a second selected reception unit.

28. The holder of claim 24, wherein two or more reception units of the holder selected from the group consisting of the first reception unit, the second reception unit, and the third reception unit are identical.

29. The holder of claim 24, wherein:
the first reception unit and the third reception unit are aligned along a shared axis; and
the second reception unit is offset from the shared axis.

30. The holder of claim 29, wherein a distance between the first reception unit and the third reception unit, is at least as large as a width of the first reception unit.

31. The holder of claim 24, wherein the first reception unit, the second reception unit, and the third reception unit are attached to each other in a linear arrangement.

32. The holder of claim 20, further comprising a support structure configured to support the holder.

33. The holder of claim 32, wherein the first reception unit is arranged between the support structure and the second reception unit.

34. The holder of claim 32, wherein the support structure is arranged adjacent to and in contact with the first reception unit.

35. The holder of claim 32, wherein the support structure is arranged adjacent to and in contact with the second reception unit.

36. The holder of claim 20, wherein the external medical module is a blood filter and the medical treatment apparatus is a blood treatment apparatus.

37. A system comprising:
a medical module;
a first holder coupled to the medical module; and
a second holder coupled to the medical module.

38. The system of claim 37, wherein the second holder is connected to the medical module and to at least one of a group consisting of a further module and an adapter for receiving a further module.

39. The system of claim 37, wherein the second holder is connected to the medical module at a position above a first reception unit of the first holder and a position below the first reception unit of the first holder.

40. The system of claim 37, further comprising a support structure, wherein the support structure is arranged to support the second holder against the first holder.

41. The system of claim 37, wherein the medical module comprises one of a group consisting of a dialysis filter and a dialyzer.

42. A system comprising:
a medical treatment apparatus;
a first holder coupled to the medical treatment apparatus; and
a second holder.

43. The system of claim 42, wherein:
the medical treatment apparatus is connected by the first holder to a first module;
the first module is connected to the second holder; and
the second holder is connected to one of a group consisting of a second module connected to a third holder and an adapter for receiving a second module, connected to a third holder.

44. The system of claim 42, wherein the medical treatment apparatus comprises one of a group consisting of an apparatus for therapeutic apheresis, an apparatus for liver support therapy, an apparatus for oxygenation, a hemofiltration apparatus, and a hemodialysis apparatus.
